(19) United States
(12) Patent Application Publication
(10) Pub. No.: US 2005/0060450 A1
(43) Pub. Date: Mar. 17, 2005

(54) Bus interface extender and method thereof

(76) Inventor: Chih-Ming Tsai, Taipei Hsien (TW)

Correspondence Address: KNOBBE MARTENS OLSON & BEAR LLP, 2040 MAIN STREET, FOURTEENTH FLOOR, IRVINE, CA, 92614, US

(21) Appl. No.: 10/804,768

(22) Filed: Mar. 19, 2004

(30) Foreign Application Priority Data
Aug. 20, 2003 (TW) 92122928

(51) Int. Cl. A61N 1/30

(52) U.S. Cl. 710/100

(57) ABSTRACT

A bus interface extender and a method thereof, is utilized between a bus arbitrator (e.g. a PCI bus arbitrator) and at least one PCI device, such that the amount of bus devices electrically coupled with the bus arbitrator can be increased without modifying architecture of the bus arbitrator. The bus arbitrator has a priority decision module, a grant decision module, and a bus signal processing module. The priority decision module determines a priority sequence for each of the bus devices. The grant decision module grants access of the proper bus device to the system bus, according to request status and priority thereof. The bus signal processing module manages transmission of request/grant signals among the PCI devices and the bus arbitrator, according to decision of the grant decision module.

### FIELD OF THE INVENTION

The present invention relates to a bus interface extender and a method thereof, and more particularly, to an extender suitable for a PCI bus arbitrator.

### BACKGROUND OF THE INVENTION

Peripheral component interconnect (PCI) bus structure is a conventional bus standard published by Intel in 1993. Of the PCI bus devices coupled to a PCI bus, network cards, sound cards and video cards are the norm. In the PCI bus structure, a PCI bus arbiter is typically used to link with several PCI bus devices within a limitation. The PCI bus arbiter plays a role for arbitrating to decide which PCI bus device is granted access to the bus when the PCI bus devices simultaneously need to use a system bus to transmit signals. These signals comprise PCI controlling signals, such as initialization device select signal (IDSEL), request signal (REQ) and grant signal (GNT).

Pins of a conventional PCI bus arbiter can only support a limited number of PCI bus devices and provide fixed functions, and thus cannot be adjusted and modified immediately. When a computer system needs more than a predetermined number of PCI bus devices, another PCI bus arbiter having more pins is needed to make a replacement and the circuit wiring of the circuit board in the computer system needs to be redistributed to meet locations of pins different from these of origin, so the cost is increased. According to the aforementioned description, it will be a work of great industry worth to design a device that can elastically expand the number of bus devices coupled to the device without modifying the original bus arbiter and the circuit.

### SUMMARY OF THE INVENTION

Therefore, an objective of the present invention is to provide a bus interface extender and a method thereof, to support more bus devices by cooperating with available bus arbitrators, such as a PCI bus arbitrator.

A bus interface extender in accordance with a preferred embodiment of the present invention is coupled between a bus arbitrator and at least one PCI device. The bus interface extender includes a priority decision module, a grant decision module and a bus signal processing module. The priority decision module determines a priority sequence for each of the bus devices. The grant decision module grants access of the proper bus device to the system bus according to priority and request status thereof. The bus signal processing module manages transmission of request/grant signals between the bus devices and the bus arbitrator according to the decision of the grant decision module.

In addition, a bus interface extending method in accordance with another preferred embodiment of the present invention is applied in a bus architecture; the bus architecture further includes a plurality of first bus devices, a plurality of second bus devices, a bus arbitrator and a bus interface extender. The first bus devices and the extender are coupled to the bus arbitrator, and the second bus devices are coupled to the bus interface extender.

When any of the first bus devices and the second bus devices needs to use a bus, the bus device will send a request signal. Conversely, whichever first bus device or second bus device receives a grant signal first is granted access to use the bus. The request signal of any first bus device is directly sent to the bus arbitrator for arbitrating, and the request signal of any second bus device is first sent to the bus interface extender for a first arbitration. The arbitration result is transmitted from the bus interface extender to the bus arbitrator for a second arbitration.

When the bus arbitrator receives several request signals simultaneously, the bus arbitrator performs an arbitration step according to a predetermined priority sequence rule and sends a grant signal to the proper first bus device or the proper bus interface extender. When the grant signal is received by the bus interface extender, the bus interface extender transmits the grant signal to the second bus device that has sent a request signal and has high priority according to another predetermined priority sequence rule.

In addition, in order to avoid the same bus device with high priority obtaining grant signals continuously and resulting in a long waiting time for other bus devices, a round-robin priority arbitration mode, for example, can be used according to the priority sequence rule of the bus interface extender of the present invention. The round-robin priority arbitration mode means that the priority sequence of a bus device can be adjusted when the bus device is granted a grant signal.

According the aforementioned description, an advantage of the present invention is that the capability of increasing the number of bus devices coupled thereto is achieved to enhance the flexibility of bus architecture design and take account of the cost concurrently without modifying the architecture of the bus arbitrator.

### BRIEF DESCRIPTION OF THE DRAWINGS

The advantages and the spirits of the present invention will become better understood by reference to the following detailed description, when taken in conjunction with the accompanying drawings, where:

FIG. 1 is a diagram illustrating a system architecture of a bus interface extender and a PCI bus arbitrator of the present invention;

FIG. 2 is a diagram illustrating an extender of the present invention;

FIG. 3 is a diagram of a priority table of a fixed-priority arbitration mode in accordance with a first preferred embodiment of the present invention;

FIG. 4A to FIG. 4B are diagrams of a round-robin priority arbitration mode in accordance with a second preferred embodiment of the present invention; and

FIG. 5 is a flow chart showing a bus interface extending method of the present invention.

### DETAILED DESCRIPTION OF THE PREFERRED EMBODIMENT

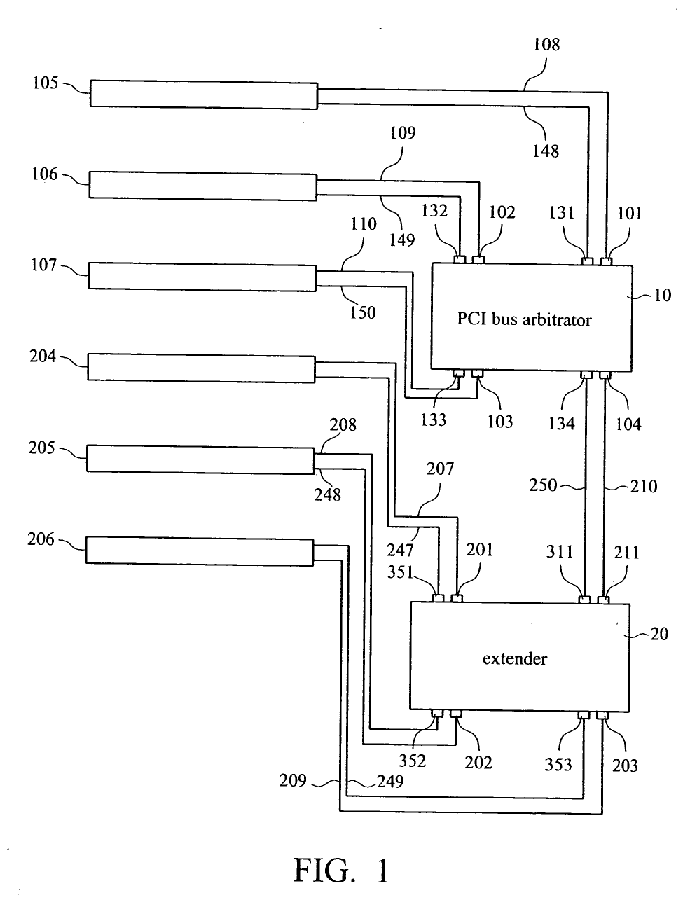

Referring to FIG. 1, FIG. 1 is a diagram illustrating a system architecture of a bus interface extender 20 and a PCI bus arbitrator 10 in accordance with a preferred embodiment of the present invention. The PCI bus arbitrator 10 works as a PCI signal access device used to arbitrate and control which PCI device coupled thereto can use the bus channel. The PCI bus arbitrator 10 includes a plurality of first pins 101-104 and 131-134 thereon, and the PCI bus arbitrator 10 can be electrically coupled to a plurality of corresponding first PCI devices 105-107 by transmitting lines 108-110 and 148-150, respectively. While the number of PCI devices that can be coupled to the original PCI bus arbitrator 10 needs to be increased, the extender 20 of the present invention can be used to connect electrically extra PCI devices. The extender 20 comprises a plurality of second pin 201-203 and 351-353 in pairs and a pair of third pins 211 and 311. The third pins 211 and 311 are coupled to two first pins 104 and 134 of the PCI bus arbitrator 10 through two transmitting lines 210 and 250, so as to transmit a request signal or a grant signal. In addition, these second pins 201-203 and 351-353 can be electrically coupled respectively to a plurality of extra second PCI devices 204-206 through transmitting lines 207-209 and 247-249.

FIG. 1 clearly illustrates that although a pair of the first pins 104 and 134 of the PCI bus arbitrator 10 is occupied for coupling the extender 20 and the decrease in the amount of PCI devices that can be coupled to the PCI bus arbitrator 10 is 1, three second PCI devices 204-206 can be additionally coupled to the PCI bus arbitrator 10 though the extender 20 of the present invention to increase the amount of the PCI devices substantially controlled by the PCI bus arbitrator 10 from four to six without redesigning circuit or replacing the original bus arbitrator with one having more pins.

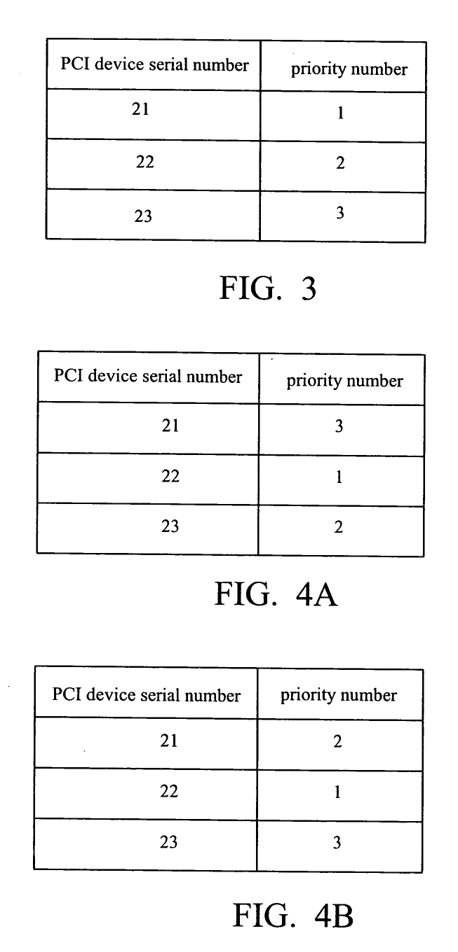

In FIG. 1, each of the first PCI devices 105-107 and the second PCI devices 204-206 is designated as a continuous but unrepeatable PCI device serial number by a system. For example, the PCI device serial numbers of the first PCI device 105, the first PCI device 106 and the first PCI device 107 are designated as 11, 12 and 13, and the PCI device serial numbers of the second PCI device 204, the second PCI device 205 and the second PCI device 206 are designated as 21, 22 and 23, as shown in the table of FIG. 3. In the system bus, these first and second PCI devices may be respectively in one of two conditions, request status and un-request status. The request status means that if any of the first or second PCI devices has a bus signal to be transmitted, it will send a request signal to ask for a formation of bus channel.

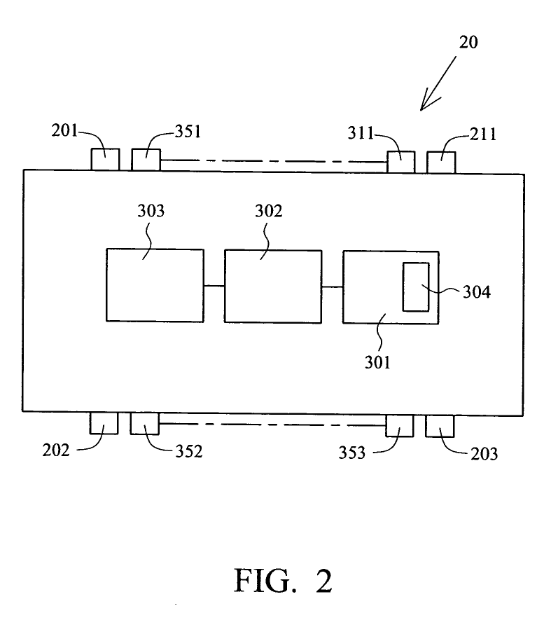

Further referring to FIG. 2, FIG. 2 is a diagram illustrating an extender 20 in accordance with a preferred embodiment of the present invention. The extender 20 mainly comprises a priority decision module 301, a grant decision module 302 and a PCI signal processing module 303. The priority decision module 301 sets a priority sequence (such as a priority table illustrated in FIG. 3) for each of the second bus devices 204-206 according to a priority decision rule 304. The priority decision rule 304 is used to decide the priority sequence for the second bus devices 204-206 to obtain the system bus controlling priority. The grant decision module 302 is used to determine the second PCI device with highest priority between these second PCI devices 204-206, and to confirm that it is the second PCI device with the highest priority in request status, when the system is initialized. The PCI signal processing module 303 is used to manage transmission of request/grant signals between the second PCI devices 204-206 and the PCI bus arbitrator 10.

Therefore, when any of second PCI devices 204-206 sends a second request signal through the transmitting line thereof to the extender 20 for an access to the bus channel, the PCI signal processing module 303 correspondingly sends a first request signal through the third pin 211 to the PCI bus arbitrator 10, and the first request signal together with the request signals sent from the first PCI devices 105-107 are arbitrated.

Subsequently, when another third pin 311 of the extender 20 receives a first grant signal transmitted from the PCI bus arbitrator 10 after arbitrating, the PCI signal processing module 303 correspondingly sends a second grant signal to the proper second PCI device and grants access of controlling right of the system bus to the proper second PCI device, so as to form the bus signal transmission between the proper second PCI device and the system bus, according to the decision of the priority sequence and request status to each of the second PCI devices 204-206 (to be described later).

It should be noted that said priority decision module 301, said grant decision module 302 and said PCI signal processing module 303 can be attained by executing circuit logic or using a controller with software or firmware. Furthermore, the priority table illustrated in FIG. 3 can be recorded in a storage medium, such as a memory, for example, a flash memory.

Referring to FIG. 3, FIG. 3 is a diagram of a priority table of a fixed-priority arbitration mode adapted in a priority decision rule 304 of an extender 20 in accordance with a first preferred embodiment of the present invention. The aforementioned priority table lists the corresponding PCI device serial number and the corresponding priority number of each of the second PCI device 204-206. The larger the PCI device serial number is, the higher the priority number is, i.e. the lower the priority sequence is. The priority sequence in the fixed-priority arbitration mode does not change, always preserving the priority of the PCI device having the PCI device serial number of 21 as the highest, the priority of the second PCI device having the PCI device serial number of 22 as the next highest, and the priority of the PCI device having the PCI device serial number of 23 as the lowest, as shown in FIG. 3.

In the first embodiment of the present invention, when the PCI signal processing module 303 of the extender 20 in FIG. 1 and FIG. 2 receives a first grant signal transmitted from the PCI bus arbitrator 10, the grant decision module 302 searches the second PCI devices 204-206 for the second PCI device with highest priority, such as the second PCI device 204 with the priority number of 1 listed in the priority table in FIG. 3, according to the priority sequence predetermined in the priority decision rule. When the PCI signal processing module 303 decides that the second PCI device 204 is not in request status, i.e. the second PCI device 204 has not sent a request signal to the extender 20 for the bus channel before, the grant decision module 302 searches for the second PCI device 205 with the second high priority (priority number is 2) according to the priority table, and simultaneously finds that the second PCI device 205 is in request status, which represents that the second PCI device 205 has sent a request signal to the PCI bus arbitrator 10 through the extender 20. The grant decision module 302 sends a second grant signal with a bus controlling right to the second PCI device 205 through the PCI signal processing module 303 to grant access by connecting the second PCI device 205 and the system bus, so as to perform PCI bus signal transaction. In contrast, if the second PCI device 205 is not in request status, the grant decision module 302 continuously searches for the second PCI device 206 with the priority number of 3 and makes a decision, and so on, to accomplish the work of arbitrating.

However, when the fixed-priority arbitration mode is adapted in the priority decision rule 304, the PCI device with higher priority usually occupies the bus controlling right. This means that if the second PCI device 204 with the highest priority is continuously in request status, all the bus controlling rights will be occupied by the second PCI device 204.

Therefore, according to a second embodiment of the present invention, the priority decision rule 304 of the extender 20 adapts a round-robin priority arbitration mode. Referring to FIG. 4A to FIG. 4B, FIG. 4A to FIG. 4B are diagrams illustrating a priority table of a round-robin priority arbitration mode adapted in the priority decision rule 304 in accordance with a second preferred embodiment of the present invention. At initialization, the priority table of the round-robin priority arbitration mode is similar to that in the first embodiment; the larger the PCI device serial number is, the lower the priority sequence is. The difference between the first embodiment and the second embodiment is that after the second PCI device with highest priority sequence obtains a first grant signal sent by the PCI bus arbitrator 10 through the extender 20, the priority of the second PCI device with highest priority sequence is lowered. In other words, the priority sequence granted to each of the second PCI devices 204-206 is changed but fixed for each arbitration. For example, on the first (or initial) arbitration illustrated in FIG. 3, the priority of the second PCI device 204 with PCI device serial number 21 is originally the highest. After the second PCI device 204 once obtains a grant signal, the priority of the second PCI device 204 becomes the lowest priority 3 shown in FIG. 4A, the priority of the second PCI device 205 with PCI device serial number 22 of the second highest shown in FIG. 3 becomes to the highest with highest priority number 1 shown in FIG. 4A on the second arbitration. At this time, the second PCI device 205 with the PCI device serial number 22 having the highest priority does not make a request, while the second PCI device 206 with the PCI device serial number 23 makes a request, so the second PCI device 206 receives a second grant signal sent by the PCI bus arbitrator 10 through the extender 20. After the second PCI device 206 has used the bus channel, the priority number of the second PCI device 206 with the PCI device serial number 23 becomes the lowest of 3, while the priority number of the second PCI device 205 with the PCI device serial number 22 remains 1 because the second PCI device 205 never transmits a PCI signal.

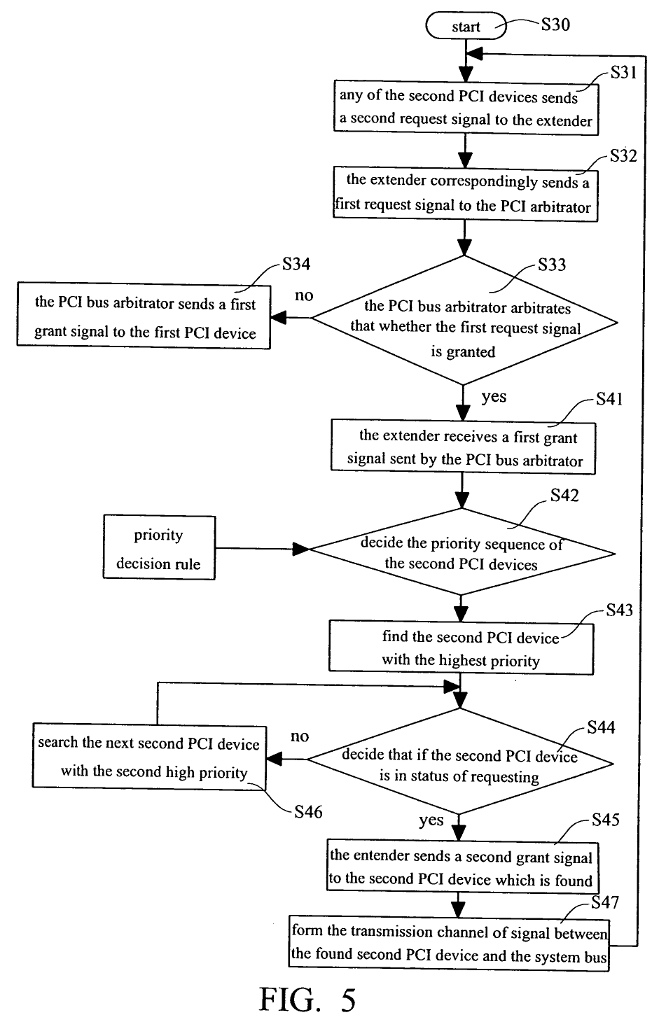

Further referring to FIG. 5, FIG. 5 is a flow chart showing a bus interface extending method in accordance with the present invention. The steps in the method are described below. First, in step S30, the system starts at an initial condition. Next, in step S31, any of the second PCI devices 204-206 sends a second request signal to the extender 20. Then, in step S32, the extender 20 correspondingly sends a first request signal to the PCI bus arbitrator 10 according to the received second request signal. After step S32, step S 33 is performed, and the PCI bus arbitrator 10 arbitrates the first request signal and the other request signals sent by the first PCI devices 105-107 to decide whether the first request signal is granted. If the result of the decision is “no”, the arbitration result represents that the request signals sent by the first PCI devices 105-107 are granted. The process then proceeds to step S34, and the PCI bus arbitrator 10 sends a first grant signal to the granted first PCI device. On the contrary, if the result of the decision is “yes”, the arbitration result represents that the first request signal sent by the extender 20 is granted. The process then proceeds to step S41, and the extender 20 receives a first grant signal sent by the PCI bus arbitrator 10. Next, step S42 is performed to decide the priority sequence of the second PCI devices 204-206 according to a specific priority decision rule. Subsequently, step S43 is performed to find the second PCI device with the highest priority. Then, step S44 is performed to confirm whether the found second PCI device is in status of requesting use of the bus.

If the result of the decision in step S44 is “yes”, step S45 is performed to correspondingly send a second grant signal to the second PCI device which is found and then step S47 is performed. On the contrary, if the result of the decision is “no”, the process proceeds to step S46 to search the next second PCI device with the second highest priority according to a specific priority decision rule, and then the process returns to step S44 to decide once more whether the second PCI device with the second high priority is in status of requesting use of the bus. If the result of the decision is still “no”, then a second PCI device with a lower priority is decided, and so on. When a second PCI device in status of requesting use of the bus is finally found and each of the other second PCI devices with the higher priority does not make a request, the process proceeds to step S45 to send a second grant signal to the second PCI device which is found, so as to grant the found second PCI device the controlling right of the system bus channel. Then, step S47 is performed to form the transmission channel of the bus signal between the second PCI device and the system bus. Subsequently, if any second PCI device makes a request, the process returns to step S31.

The priority calculation of the extender of the present invention is not limited to the aforementioned fixed-priority arbitration mode calculation and the round-robin priority arbitration mode calculation, so the extender of the present invention can be coupled with a conventional PCI bus arbitrator. Therefore, the amount of PCI devices that can be controlled by the conventional PCI bus arbitrator can be increased without redesigning the circuit or using another PCI bus arbitrator having more pins to replace the original one.

As is understood by a person skilled in the art, the foregoing preferred embodiments of the present invention are illustrated of the present invention rather than limiting of the present invention. It is intended to cover various modifications and similar arrangements included within the spirit and scope of the appended claims, the scope of which should be accorded the broadest interpretation so as to encompass all such modifications and similar structure.

1. A bus interface extender used to increase an amount of bus devices that can be controlled by a bus arbitrator, wherein the bus arbitrator includes a plurality of first pins, and each of the first pins can be electrically coupled to a corresponding first bus device, so the bus arbitrator arbitrates request signals asking for use of a bus channel sent by the first bus devices, the bus interface extender including:
a plurality of second pins; and
at least one third pin, wherein the at least one third pin is electrically coupled to a corresponding one of the first pins of the bus arbitrator, and each of the second pins can be electrically coupled to a corresponding second bus device, so the bus interface extender arbitrates each request signal sent by each second bus device through the second pins asking for use of a bus channel, according to a first grant signal, that allows the use of the bus channel and is produced after arbitrating by the bus arbitrator and received by the bus interface extender through the at least one third pin.

2. The bus interface extender according to claim 1, further comprising:
a priority decision module for determining a priority sequence in using the bus channel for each second bus device according to a priority decision rule;
a grant decision module for deciding one of the second bus devices with the highest priority according to the priority decision rule and confirming whether the second bus device with the highest priority is in request status; and
a bus signal processing module for correspondingly sending a second grant signal to a proper one of the second bus device according to the first grant signal and a decision result decided by the grant decision module to form signal transmission between the proper one of the second bus device and a system bus.

3. The bus interface extender according to claim 2, wherein the priority decision rule uses a fixed-priority arbitration mode.

4. The bus interface extender according to claim 2, wherein the priority decision rule uses a round-robin priority arbitration mode.

5. The bus interface extender according to claim 1, wherein the bus arbitrator and the bus interface extender are installed in a PCI bus architecture.

6. The bus interface extender according to claim 1, wherein at least one of the second bus devices sends a request signal of asking use of the bus channel to the bus arbitrator through the at least one third pin of the bus interface extender, and the request signal asking for use of the bus channel is arbitrated by the bus arbitrator.

7. A bus interface extender used to electrically couple to a bus arbitrator to increase an amount of bus devices that can be controlled by the bus arbitrator, wherein at least one first bus device is coupled to the bus arbitrator directly, and at least one second bus device is electrically coupled to the bus interface extender, so that the bus arbitrator can arbitrate any request signals sent from each first bus device and each second bus device through the bus interface extender, the bus interface extender including:
a priority decision module for determining a priority sequence in using a bus channel for each the at least one second bus device according to a priority decision rule;
a grant decision module for deciding one of the at least one second bus device with the highest priority according to the priority decision rule, and the at least one second bus device with the highest priority has sent a request signal; and
a bus signal processing module for correspondingly sending a second grant signal to the at least one second bus device with the highest priority according to the first grant signal and a decision result decided by the grant decision module to form signal transmission between the at least one second bus device with the highest priority and a system bus.

8. The bus interface extender according to claim 7, wherein the priority decision rule uses a fixed-priority arbitration mode.

9. The bus interface extender according to claim 7, wherein the priority decision rule uses a round-robin priority arbitration mode.

10. A bus interface extending method, using an extender to increase an amount of bus devices controllable by a bus arbitrator, wherein the bus arbitrator is electrically coupled to at least one first bus device directly, and the extender is electrically coupled to at least one second bus device, so that the bus arbitrator can arbitrate any request signals sent from each first bus device and each second bus device through the extender, bus interface extending method comprising:
receiving a first grant signal after arbitrating through the extender;
searching for one having the highest priority from the at least one second bus device according to a priority sequence of each second bus device predetermined in a priority decision rule, wherein the at least one second bus device with the highest priority has sent a request signal; and
sending a second grant signal to the at least one second bus device with the highest priority by the extender to grant access of forming a channel for controlling signal transmission between the at least one second bus device with the highest priority and a system bus.

11. The bus interface extending method according to claim 10, wherein the bus arbitrator is installed in a PCI bus architecture.

12. The bus interface extending method according to claim 10, wherein the priority decision rule uses a fixed-priority arbitration mode.

13. The bus interface extending method according to claim 10, wherein the priority decision rule uses a round-robin priority arbitration mode.

14. A bus system, electrically coupled to a plurality of first bus devices and a plurality of second bus devices, the bus system comprising:
a bus arbitrator coupled to the first bus devices and an extender for arbitrating a request signal sent by any of the first bus devices and the extender; and
the extender electrically coupled between the bus arbitrator and the second bus devices for transmitting a request signal sent by any of the second bus devices to the bus arbitrator for arbitrating, and when the extender receives a grant signal produced by the bus arbitrator after arbitrating, the extender transmits the grant signal to a proper one of the second bus devices according to a priority sequence and request status of the second bus devices, so as to form a signal transmission channel between the proper one of the second bus devices and a system bus.

15. The bus system according to claim 14, wherein the bus system is a PCI bus system.

16. The bus system according to claim 15, wherein the extender includes a memory medium and a circuit logic, the memory medium is used to store the priority sequence, and the circuit logic is used to determine grant of access to the grant signal to a corresponding one of the second bus devices according to the memory medium.

17. The bus system according to claim 16, wherein the circuit logic adjusts the priority sequence in the memory medium as each of the second bus devices is granted the grant signal.

18. A bus device extending method, comprising:
coupling a plurality of first bus devices to a bus arbitrator;
coupling a plurality of second bus devices to an extender;
coupling the extender to the bus arbitrator;
sending a first request signal from the extender to the bus arbitrator correspondingly when the extender receives a second request signal sent by the second bus devices; and
sending a second grant signal from the extender to a proper one of the second bus devices correspondingly according to a priority sequence of the second bus devices when the extender receives a first grant signal sent from the bus arbitrator.

19. The bus device extending method according to claim 18, wherein the priority sequence of each second bus device is fixed.

20. The bus device extending method according to claim 18, wherein the priority sequence is changed and adjusted with a sequence when each second bus device is granted the second grant signal.

21. The bus device extending method according to claim 18, wherein the bus arbitrator is a PCI bus arbitrator.
